(19) United States
(12) Patent Application Publication
(10) Pub. No.: US 2026/0160771 A1
(43) Pub. Date: Jun. 11, 2026

(54) CREATION METHOD FOR CALIBRATION CURVE, FLUORESCENCE POLARIZATION IMMUNOASSAY METHOD, FLUORESCENCE POLARIZATION IMMUNOASSAY DEVICE, AND CALIBRATION CURVE CREATION KIT

(71) Applicant: TIANMA JAPAN, LTD., Kawasaki (JP)

(72) Inventors: Ayuko IMAI, Kawasaki (JP); Koji SHIGEMURA, Kawasaki (JP)

(73) Assignee: TIANMA JAPAN, LTD., Kawasaki (JP)

(21) Appl. No.: 19/179,194

(22) Filed: Apr. 15, 2025

(30) Foreign Application Priority Data
Apr. 22, 2024 (JP) 2024-068957
Dec. 25, 2024 (JP) 2024-229081

(51) Int. Cl. G01N 33/96; G01N 33/53

(52) U.S. Cl. CPC G01N 33/96; G01N 33/5308; G01N 2496/00

(57) ABSTRACT

A creation method for a calibration curve uses an antibody that has bindability to a target substance, and a fluorescently labeled substance in which the target substance is labeled with a fluorescent dye. The calibration curve is created on the basis of a degree of fluorescence polarization P₀ of a first reference sample that does not contain the target substance, and the degree of fluorescence polarization P_{cone} of a calibration curve creation sample_{conc} in which the concentration of the added target substance exceeds a concentration C_{Sα} of the target substance.

### CROSS-REFERENCE TO RELATED APPLICATIONS

This application claims the benefit of Japanese Patent Application No. 2024-68957, filed on Apr. 22, 2024, and Japanese Patent Application No. 2024-229081, filed on Dec. 25, 2024, of which the entirety of the disclosures is incorporated by reference herein.

### FIELD OF THE INVENTION

The present disclosure relates generally to a creation method for a calibration curve, a fluorescence polarization immunoassay method, a fluorescence polarization immunoassay device, and a calibration curve creation kit.

### BACKGROUND OF THE INVENTION

One immunoassay that utilizes an antigen-antibody reaction is a measurement method called “fluorescence polarization immunoassay” (FPIA). In this method, the concentration of a target substance is estimated by fluorescence polarization measurement. There are two types of FPIA: competitive and noncompetitive. Of these types, in competitive FPIA, the target substance and a fluorescently labeled target substance (tracer) are caused to compete to react with an antibody. The tracers not bound to the antibodies move vigorously in solution and fluorescence is randomly emitted even when irradiated with polarized excitation light. Meanwhile, the tracers bound to the antibodies are less likely to move and, as such, fluorescence biased in the polarization direction of the excitation light is emitted.

In competitive FPIA, fluorescence intensity in a direction parallel to the polarization direction of the excitation light and fluorescence intensity in a direction perpendicular to the polarization direction of the excitation light are each obtained, and the degree of bias of the fluorescence intensity in both directions is measured as the degree of fluorescence polarization. This degree of fluorescence polarization is dependent on the amount of tracer-antibody conjugates and, as such, it is possible to measure the concentration of the target substance by utilizing the degree of fluorescence polarization as an index.

In FPIA, a calibration curve expressing the relationship between the measured degree of fluorescence polarization and the concentration of the target substance is created and, from the obtained calibration curve, the concentration of the target substance contained in the sample to be measured is measured (for example, see Unexamined Japanese Patent Application Publication No. 2011-47802 and the description of US Patent Application Publication No. 2009/0023595).

Pure water is an example of a diluent that is used when creating the calibration curve. Since pure water is readily available, the calibration curve can be efficiently created by using pure water as the diluent.

However, when the sample to be measured contains contaminants that affect the reaction between the target substance and the antibody, when using pure water as a diluent, it is difficult to create a calibration curve that takes into account the effects of the contaminants.

One example of a method for creating a calibration curve that takes into account the effects of contaminants in the sample to be measured is a method that uses the sample to be measured itself as the diluent, instead of pure water. That is, it is thought that a calibration curve that takes into account the effects of contaminants can be created by adding a known amount of the target substance to a plurality of samples to be measured to prepare calibration curve creation samples.

However, the degree of fluorescence polarization of a low-concentration region of the calibration curve created using such a method is easily affected by the target substance originally contained in the sample to be measured and, consequently, there is a need for further improvement of this method in order to create a calibration curve that is accurate in a wide range from the low-concentration region to a high-concentration region.

Specifically, when the amount of the target substance contained in the sample to be measured is a small amount, it is possible to create a calibration curve using all of the prepared calibration curve creation samples by adjusting the tracer amount and the antibody amount (in other words, in a case in which a degree of fluorescence polarization P_{dil} is a value equivalent to a degree of fluorescence polarization P₀ in the calibration curve creation method of the present disclosure, in the creation method for a calibration curve of the present disclosure, it is possible to create a calibration curve using the degree of fluorescence polarization P_{dil} instead of the degree of fluorescence polarization P₀). However, as described above, this method can only be used when the amount of the target substance contained in the sample to be measured is a small amount.

### SUMMARY OF THE INVENTION

A method for creating a calibration curve according to a first aspect of the present application is a method for creating a calibration curve for a fluorescence polarization immunoassay that uses an antibody that has bindability to a target substance and a fluorescently labeled substance in which the target substance is labeled with a fluorescent dye, the method including:
a step 1a of preparing a first reference sample that does not contain the target substance;
a step 1b, after the step 1a, of adding the antibody and the fluorescently labeled substance to the first reference sample and measuring a degree of fluorescence polarization P₀ of the first reference sample;
a step 1c of separating, from a sample to be measured α that contains the target substance at a concentration C_{Sα}, samples to be measured α1 to αp in which the number of samples is an integer p of 3 or greater, adding different amounts of the target substance to each of the samples to be measured al to ap, and preparing p calibration curve creation samples 1 to p including a calibration curve creation sample_{conc} in which a concentration of the target substance added later exceeds the original concentration C_{Sα} of the target substance;
a step 1d, after the step 1c, of adding the antibody and the fluorescently labeled substance, in amounts equal to the added amounts in step 1b, to each of the calibration curve creation samples 1 to p, and measuring degrees of fluorescence polarization P₁ to P_{P} of the respective calibration curve creation samples 1 to p; and
a step 1e, after the step 1b and the step 1d, of creating, based on the degree of fluorescence polarization P₀ and a degree of fluorescence polarization P_{cone} of the calibration curve creation sample_{conc} among the calibration curve creation samples 1 to p, a calibration curve expressing a relationship between an amount of the target substance and the degree of fluorescence polarization of the sample.

A fluorescence polarization immunoassay method according to a second aspect of the present application is a fluorescence polarization immunoassay method in which an antibody, that has bindability to a target substance, and a fluorescently labeled substance, in which the target substance is labeled with a fluorescent dye are used to measure a concentration C_{Sβ} of the target substance in a sample to be measured β, the method including:
a step 4a of adding the antibody and the fluorescently labeled substance, in amounts equal to the added amounts in the step 1b, to the sample to be measured β, and measuring a degree of fluorescence polarization P_{Xβ} of the sample to be measured β; and
a step 4b, after the step 4a, of deriving, based on the degree of fluorescence polarization P_{Xβ}, the concentration C_{Sβ} of the target substance from the calibration curve created by the method according to the first aspect.

A program according to a third aspect of the present application is a program used in the fluorescence polarization immunoassay method according to the second aspect, the program causing a computer to execute:
a step of deriving, based on the degree of fluorescence polarization P_{Xβ} of the sample to be measured β, the concentration C_{Sβ} of the target substance from the calibration curve created by the method according to the first aspect.

A fluorescence polarization immunoassay device according to a fourth aspect of the present application is a fluorescence polarization immunoassay device that uses an antibody, that has bindability to a target substance, and a fluorescently labeled substance, in which the target substance is labeled with a fluorescent dye to measure a concentration C_{Sβ} of the target substance in a sample to be measured β, the device including:
an emission optical system that emits linearly polarized excitation light on a sample;
a polarized light adjustment element that selectively allows, of fluorescence emitted from the sample, a linearly polarized component corresponding to a drive signal to pass through;
a receiver that detects fluorescence intensity that has passed through the polarized light adjustment element; and
a controller that outputs the drive signal to the polarized light adjustment element and measures, in accordance with the drive signal and based on the fluorescence intensity detected by the receiver, a degree of polarization of the sample,
wherein
the controller includes a storage medium that stores a program used in the fluorescence polarization immunoassay method according to the second aspect, the program causing a computer to execute:
a step of deriving, based on the degree of fluorescence polarization P_{Xβ} of the sample to be measured β, the concentration C_{Sβ} of the target substance from the calibration curve.

A calibration curve creation kit according to a fifth aspect of the present application is a calibration curve creation kit including:
an antibody that has bindability to a target substance; and a fluorescently labeled substance in which the target substance is labeled with a fluorescent dye;
and further including:
at least one of a means for removing the target substance from a solution containing the target substance, and a liquid not containing the target substance.

It is to be understood that both the foregoing general description and the following detailed description are exemplary and explanatory and are not restrictive of this disclosure.

In the present disclosure, “a calibration curve” sometimes means “a function that represents a calibration curve”, and “creating a calibration curve” includes not only creating a graph that allows visual recognition of the relationship between an amount of the target substance and the degree of fluorescence polarization of the sample, but also deriving a function that represents a calibration curve.

In the present disclosure, the degree of fluorescence polarization such as the degree of fluorescence polarization P₀, the degree of fluorescence polarization P_{conc}, and the like is P_{A} calculated by Equation (I) below or P_{CB} calculated by Equation (II) below.

$${P}_{A} = \frac{A ( {I}_{∥} ) - A ( {I}_{⊥} )}{A ( {I}_{∥} ) + A ( {I}_{⊥} )} \qquad ( I )$$

In Equation (I), A (In) is the fluorescence intensity of fluorescence having a polarized light component parallel to an excitation polarization direction when a fluorescently labeled substance and an antibody are added to the sample and measured. A(I_{⊥}) is the fluorescence intensity of fluorescence having a polarized light component perpendicular to the excitation polarization direction when the fluorescently labeled substance and the antibody are added to the sample and measured.

$${P}_{CB} = \frac{( C ( {I}_{∥} ) - B ( {I}_{∥} ) ) - ( C ( {I}_{⊥} ) - B ( {I}_{⊥} ) )}{( C ( {I}_{∥} ) - B ( {I}_{∥} ) ) + ( C ( {I}_{⊥} ) - B ( {I}_{⊥} ) )} \qquad ( II )$$

In Equation (II), B(I_{II}) is the fluorescence intensity of fluorescence having a polarized light component parallel to the excitation polarization direction when the sample is measured without modification. B(I_{⊥}) is the fluorescence intensity of fluorescence having a polarized light component perpendicular to the excitation polarization direction when the sample is measured without modification.

C(I_{II}) is the fluorescence intensity of fluorescence having a polarized light component parallel to the excitation polarization direction when the fluorescently labeled substance and the antibody are added to the sample and measured. C(I_{⊥}) is the fluorescence intensity of fluorescence having a polarized light component perpendicular to the excitation polarization direction when the fluorescently labeled substance and the antibody are added to the sample and measured.

When the sample contains an autofluorescent substance, P_{A} calculated by Equation (I) is affected by the fluorescence emitted by the autofluorescent substance and, as such, P_{A} is not suitable as calibration curve creation data.

Meanwhile, as in Equation (II), in a corrected degree of fluorescence polarization (degree of fluorescence polarization P_{CB}) calculated using the measurement data of the sample containing the fluorescently labeled substance and the antibody and measurement data of a sample not containing these components, the effects of the fluorescence emitted by the autofluorescent substance are eliminated and, as such, the corrected degree of fluorescence polarization is suitable as calibration curve creation data.

Examples of cases in which it is preferable to use the corrected degree of fluorescence polarization calculated by Equation (II) include, for example, cases in which the sample to be measured is a beverage, a food, or the like that contains an autofluorescent substance such as certain types of vitamins and the like and, as discussed later, cases in which the sample to be measured is subjected to a specific enzyme treatment in order to remove the target substance from the sample to be measured.

Thus, in the creation method for a calibration curve of the present disclosure, it is preferable that P_{A} calculated by Equation (I) and P_{CB} calculated by Equation (II) be used appropriately in consideration of the effects of the fluorescence emitted by the autofluorescent substance in the sample.

### BRIEF DESCRIPTION OF DRAWINGS

A more complete understanding of this application can be obtained when the following detailed description is considered in conjunction with the following drawings, in which:

FIG. 1 is a schematic drawing illustrating the configuration of a fluorescence polarization immunoassay device 1;

FIG. 2 is a schematic drawing of a microdevice 23;

FIG. 3 is a drawing explaining a preferable concentration of a calibration curve creation sample;

FIG. 4 is a drawing illustrating a creation process of a calibration curve (steps 1a to 1b);

FIG. 5 is a drawing illustrating a creation process of the calibration curve (steps 1c to 1d);

FIG. 6 is a drawing illustrating a creation process of the calibration curve (step 1e);

FIG. 7 is a drawing illustrating a process of an additional measurement 1 (steps 2a to 2c);

FIG. 8 is a drawing illustrating a process of the additional measurement 1 (step 2d);

FIG. 9 is a drawing illustrating a process of the additional measurement 1 (steps 2e to 2f);

FIG. 10 is a drawing illustrating a process of an additional measurement 2 (steps 3a to 3d);

FIG. 11 is a drawing illustrating a process of the additional measurement 2 (steps 3e to 3g);

FIG. 12 is a drawing illustrating a process of the additional measurement 2 (step 3h);

FIG. 13 is a drawing illustrating a creation process of a calibration curve (steps 1a to 1b);

FIG. 14 is a drawing illustrating a creation process of the calibration curve (steps 1c to 1d);

FIG. 15 is a drawing illustrating a creation process of the calibration curve (step 1e);

FIG. 16 is a calibration curve created in Example 1;

FIG. 17 is a calibration curve created in Example 2;

FIG. 18 is a calibration curve created in Example 6;

FIG. 19 is a calibration curve created in Example 7; and

FIG. 20 is a calibration curve created in Example 8.

### DETAILED DESCRIPTION OF THE INVENTION

### 1. Creation Method for Calibration Curve

### Sample to be Measured

A calibration curve created by the present disclosure is used when measuring a concentration Cs of a target substance contained in a sample to be measured (hereinafter may be abbreviated as “concentration Cs of target substance”). Examples of the sample to be measured include beverages, foods, washing fluid, tissue extracts, cell extracts, cell culture supernatants, blood, saliva, urine, lymph fluid, and the like.

### Target Substance

The target substance is a substance for which the concentration is to be measured by the calibration curve created by the present disclosure. The target substance is a compound, at least part of which can be used as an epitope to prepare an antibody. Examples of the target substance include antigens, haptens, and the like. Specific examples thereof include proteins, glycoproteins, peptides, polypeptides, oligonucleotides, polynucleotides, antibodies, hormones, drugs, enzymes, receptors, and the like.

Note that, although haptens bind to antibodies, they have a small molecular weight and, consequently, are a substance that does not exhibit immungenicity, which is the activity of inducing antibody production, on its own. As such, when the target substance is a hapten, as described later, the hapten is caused to bind to an immunogenic substance such as a protein or the like to form an immunogenic complete antigen, thereby making it possible to measure the concentration in the sample to be measured.

Examples of the haptens include histamine, γ-aminobutyric acid (GABA), dopamine, thyroid hormones, steroid hormones, and the like.

Examples of the immunogenic substance include immunogenic proteins, polypeptides, carbohydrates, polysaccharides, lipopolysaccharides, nucleic acids, and the like. Among these, polypeptides or proteins such as bovine serum albumin (BSA), keyhole limpet hemocyanin (KLH), thyroglobulin, and the like are preferable.

### Antibody

The antibody used in the present disclosure is an antibody that can bind to the target substance. Specifically, the antibody used in the present disclosure has an ability to recognize and bind to at least a portion of the target substance as an epitope. Examples of the antibody include monoclonal antibodies, multispecific antibodies, bifunctional antibodies, human antibodies, humanized antibodies, antibodies derived from birds such as chickens, mammals such as cows and camels, and other animals, recombinant antibodies, chimeric antibodies, single chain Fv (“scFv”), single chain antibodies, single domain antibodies, Fab fragments, F(ab′) fragments, F(ab′)2 fragments, disulfide-linked Fv (“sdFv”), anti-idiotypic (“anti-Id”) antibodies, dual domain antibodies, dual variable domain antibodies, and the like.

When the target substance is a hapten, typically, an antibody is used in which a hapten derivative, in which an immunogenic substance is bound to the hapten via a linker, is the immunogen. The linker is a group of atoms introduced between the immunogenic substance and the hapten. Examples of the linker include groups that have an acid amide bond, and the like.

### Fluorescently Labeled Substance

The fluorescently labeled substance is a compound in which the target substance is labeled by a fluorescent dye.

The fluorescent dye is a dye that emits fluorescence. Each type of fluorescent dye has its own fluorescence lifetime. In the present disclosure, in accordance with the molecular weight and the like of the target substance, it is possible to appropriately select and use a fluorescent dye having a fluorescence lifetime of 1 to 10 nanoseconds, a fluorescent dye having a fluorescence lifetime of more than 10 nanoseconds to 200 nanoseconds, and a fluorescent dye having a fluorescence lifetime of more than 200 nanoseconds to 3,000 nanoseconds. Examples of the fluorescent dye having a fluorescence lifetime of 1 to 10 nanoseconds include indolenines; fluorescein compounds such as chlorotriazinylaminofluorescein, 4′-aminomethylfluorescein, 5-aminomethylfluorescein, 6-aminomethylfluorescein, 6-carboxyfluorescein, 5-carboxyfluorescein, 5-aminofluorescein, 6-aminofluorescein, thioureafluorescein, and methoxytriazinylaminofluorescein; rhodamine derivatives such as rhodamine B, rhodamine 6G, and rhodamine 6GP; and, as registered trademarks or product names, Alexa Fluor 488 and other Alexa Fluor series, the BODIPY series, the DY series, the ATTO series, the Dy Light series, the Oyster series, the HiLyte Fluor series, Pacific Blue, Marina Blue, Acridine, Edans, Coumarin, DANSYL, FAN, Oregon Green, Rhodamine Green-X, NBD-X, TET, JOE, Yakima Yellow, VIC, HEX, R6G, Cy3, TAMRA, Rhodamine Red-X, Redmond Red, ROX, Cal Red, Texas Red, LC Red 640, Cy5, Cy5.5, and LC Red 705. Examples of the fluorescent dye having a fluorescence lifetime of more than 10 nanoseconds to 200 nanoseconds include naphthalene derivatives such as dialkylaminonaphthalenesulfonyl; and pyrene derivatives such as N-(1-pyrenyl) maleimide, aminopyrene, pyrenebutanoic acid, and alkynylpyrene. Examples of the fluorescent dye having a fluorescence lifetime of more than 200 nanoseconds to 3,000 nanoseconds include metal complexes containing platinum, rhenium, ruthenium, osmium, europium, and other metals.

Examples of a method for labeling the target substance with the fluorescent dye include a method of directly binding the fluorescent dye to the target substance, and a method of binding the fluorescent dye to the target substance via an appropriate linker such as an oligoethylene glycol, an alkyl chain, or the like. In these methods, substituents such as carboxyl groups, amino groups, hydroxyl groups, thiol groups, and phenyl groups contained in the fluorescent dye and/or target substance can be used.

### Fluorescence Polarization Immunoassay Method

A fluorescence polarization immunoassay method is a measurement method that utilizes the competitive reaction of the substances, and a change in the degree of fluorescence polarization caused by a change in the molecular weight of the competing substances. In a case in which fluorescent molecules in a liquid are irradiated with plane polarized light as excitation light, when the fluorescent molecules are not moving much, the fluorescent molecules emit polarized fluorescence in the same plane as the excitation plane. Meanwhile, when the fluorescent molecules rotate due to Brownian motion in an excited state, the fluorescent molecules emit polarized fluorescence in a plane different from the excitation plane and, as such, the degree of fluorescence polarization decreases overall. Thus, the degree of fluorescence polarization is a physical property related to the degree to which the fluorescent molecules rotate from when being excited to when emitting fluorescence. Additionally, the size (molecular weight) of the fluorescent molecules affects the degree to which the fluorescent molecules rotate. For example, low molecular weight molecules such as the free fluorescently labeled substance rotate vigorously due to Brownian motion in a solution, but high molecular weight molecules such as the fluorescently labeled substance to which the antibody is bonded do not exhibit vigorous movement. Accordingly, it is possible to obtain information related to the change in molecular weight of the fluorescently labeled substance in the solution by measuring the degree of fluorescence polarization. Furthermore, it is possible to measure the concentration Cs of the target substance in the sample to be measured by the following method.

In a solution in which a target substance A, an antibody B that can bind to the target substance A, and a fluorescently labeled substance C in which the target substance A is labeled with the fluorescent dye are mixed, the target substance A, the antibody B, and the fluorescently labeled substance C competitively react in the solution and, as such, when the concentration of the target substance A is high, the amount of conjugates of the target substance A and the antibody B increases and, as a result, the amount of the free fluorescently labeled substance C increases. Thus, the concentration of the target substance A affects the amount of the free fluorescently labeled substance C (or the fluorescently labeled substance C bound to the antibody B) and, as such, it is possible to measure the concentration of the target substance A by creating a calibration curve that expresses the relationship between the concentration of the target substance A and the degree of fluorescence polarization.

### Fluorescence Polarization Immunoassay Device

In the creation method for a calibration curve of the present disclosure, the degree of fluorescence polarization of various samples is measured. The measurement device or measurement equipment used to measure the degree of fluorescence polarization is not particularly limited, but the degree of fluorescence polarization can be efficiently measured by using a microdevice.

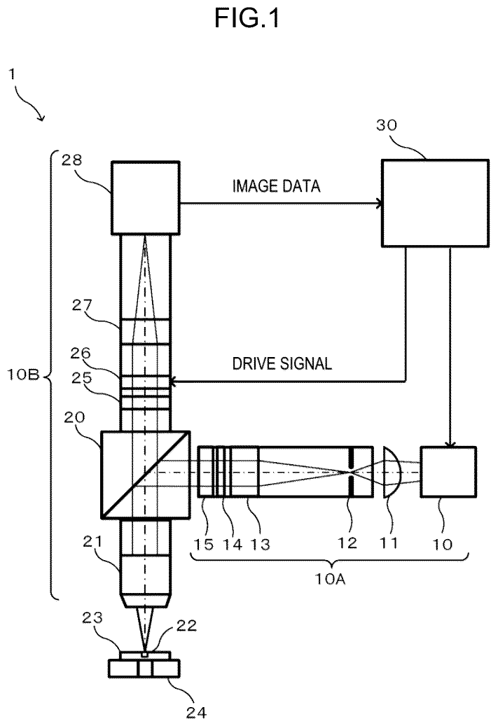

FIG. 1 illustrates an example of a fluorescence polarization immunoassay device that uses a microdevice.

The fluorescence polarization immunoassay device 1 includes a light source 10, a condenser lens 11, an iris 12, a collimator 13, a polarization element 14, an excitation light filter 15, and a dichroic mirror 20. The fluorescence polarization immunoassay device 1 includes an objective lens 21, a microdevice 23 on which a sample 22 is placed, a stage 24, an absorption filter 25, a polarized light adjustment element 26, an imaging lens 27, an imaging element 28, and a controller 30.

In one example, the light source 10 is implemented as a light emitting diode, and emits excitation light of a wavelength (for example, blue light having a central wavelength of 470 nm) that excites the fluorescence of the sample. The excitation light from the light source 10 is focused by the condenser lens 11 and passes through the iris 12. The iris 12 reduces the intrusion of external light other than the excitation light.

The excitation light that has passed through the iris 12 is converted to parallel light by the collimator 13, and enters the polarization element 14. The polarization element 14 is, for example, a polarizing plate, a polarizing beam splitter, or a liquid crystal cell and, in this case, is a polarizing plate. The polarization element 14 allows light that is linearly polarized in a specific direction to pass through. The linearly polarized excitation light from the polarization element 14 passes through the excitation light filter 15. The excitation light filter 15 is a filter that selects a wavelength range including the wavelength of the excitation light, and reduces light of wavelengths different from that of the excitation light from the polarization element 14. The dichroic mirror 20 reflects the excitation light that has passed through the excitation light filter 15 toward the objective lens 21.

The objective lens 21 focuses the linearly polarized excitation light, reflected by the dichroic mirror 20, on the sample 22 accommodated in the microdevice 23 on the stage 24. The sample 22 generates fluorescence of a specific wavelength (for example, green light) in accordance with the linearly polarized excitation light from the objective lens 21. The fluorescence becomes parallel light at the objective lens 21, and passes through the dichroic mirror 20 and the absorption filter 25. The dichroic mirror 20 selectively allows light of a specific wavelength region including the fluorescence from the sample 22 to pass through, and reflects other light. The absorption filter 25 is a filter that selects a wavelength range including the wavelength of the fluorescence from the sample 22, and reduces light other than the fluorescence.

The fluorescence that has passed through the absorption filter 25 enters the polarized light adjustment element 26. In one example, the polarized light adjustment element 26 is implemented as a polarizing plate, a polarizing beam splitter, or a liquid crystal cell. The polarized light adjustment element 26 may be implemented as a polarization filter in a polarization camera. A polarization camera is an imaging device in which a polarization filter is mounted on a sensor and thereby acquires polarization information of a subject. In the following description, the polarized light adjustment element 26 is a liquid crystal cell controlled by a drive signal (applied voltage). The polarized light adjustment element 26 can adjust a transmitted light intensity of the linearly polarized component. Specifically, the polarized light adjustment element 26 can adjust the transmitted light intensity of the linearly polarized light parallel to the polarization direction of the excitation light and the transmitted light intensity of the linearly polarized light perpendicular to the polarization direction of the excitation light. Furthermore, the polarized light adjustment element 26 can adjust the transmitted light intensity of light polarized in a direction corresponding to the drive signal, described later.

The fluorescence of the linearly polarized light that has passed through the polarized light adjustment element 26 enters an imaging plane of the imaging element 28 via the imaging lens 27. The surface of the sample 22 and the imaging plane of the imaging element 28 are in an imaging relationship. The imaging element 28 includes, for example, a charge coupled device (CCD) or a complementary metal oxide semiconductor (CMOS) image sensor that includes a plurality of pixels. The imaging element 28 generates image data corresponding to the fluorescence intensity generated by the sample 22, and sends the generated image data to the controller 30.

The controller 30 performs overall control of the entire fluorescence polarization immunoassay device 1. Specifically, the controller 30 controls the light source 10, the polarized light adjustment element 26, and the imaging element 28. The controller 30 acquires a fluorescence image imaged by the imaging element 28.

For example, during measurement operation, the controller 30 causes the light source 10 to emit the excitation light on the sample 22. The controller 30 uses a DA converter (not illustrated) to output the drive signal to the polarized light adjustment element 26. By outputting the drive signal to the polarized light adjustment element 26, the controller 30 can control the polarized light component of the fluorescence that passes through the polarized light adjustment element 26.

In one example, the polarized light adjustment element 26 includes two transparent substrates that face each other, a transparent electrode disposed on opposing surfaces of the substrates, liquid crystal material sealed between the substrates, and a polarizing plate disposed on an outside surface on the imaging device side (exit side or downstream side) of the polarized light adjustment element 26. The configuration of the polarized light adjustment element 26 may be determined as desired provided that it is possible to adjust the polarized light component of the fluorescence that passes through the polarized light adjustment element 26.

The constituents of the fluorescence polarization immunoassay device 1 described above are summarized as follows.
(A) An emission optical system 10A that includes the light source 10, the condenser lens 11, the iris 12, the collimator 13, the polarization element 14, and the excitation light filter 15, and that emits the linearly polarized excitation light on the sample 22;
(B) The microdevice 23 that accommodates the sample 22, and the stage 24 on which the microdevice 23 is placed;
(C) An observation optical system 10B that includes the polarized light adjustment element 26 that adjusts, of the fluorescence emitted from the sample 22 and in accordance with the drive signal form the controller 30, the transmitted linearly polarized component, and the imaging element 28 that images the fluorescence image that transmits through the polarized light adjustment element 26;
(D) The controller 30 that functions as a driver that outputs the drive signal to the polarized light adjustment element 26, detects the fluorescence intensity on the basis of the fluorescence image imaged by the imaging element 28, and measures the degree of polarization of the sample 22 in accordance with the drive signal to analyze the sample.

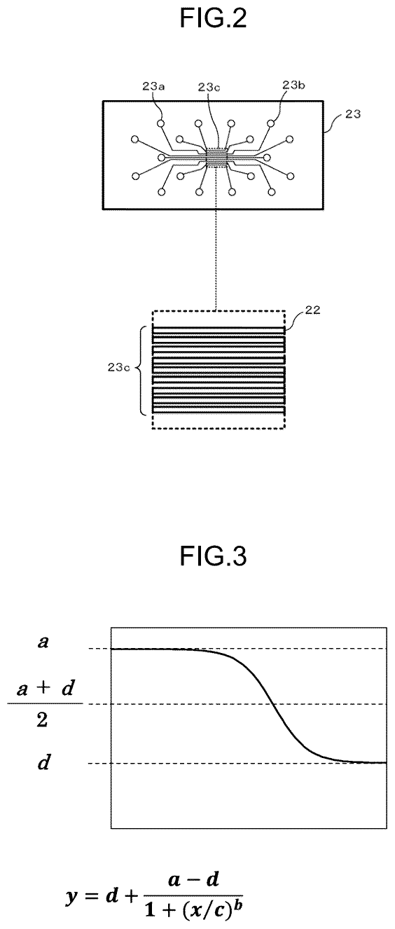

As illustrated in FIG. 2, the microdevice 23 includes a plurality of channels 23c that each include one end that is connected to an injection port 23a and another end that is connected to a discharge port 23b. A measurement liquid including a calibration curve creation sample or a sample to be measured can be individually supplied as the sample 22 to the plurality of channels 23c. The channels 23c and the imaging plane of the imaging element 28 are in an imaging relationship and, as such, if the plurality of channels 23c are imaged by the imaging element 28, fluorescence images of the plurality of samples 22 supplied to the plurality of channels 23c can be obtained at once, and the degree of polarization of each sample 22 can be measured at once. This measurement is performed on the basis of the fluorescence intensity of a region of interest (ROI) of the image data corresponding to the channels 23c in the fluorescence image.

### Creation Method for Calibration Curve

The creation method for a calibration curve of the present disclosure is a method for creating a calibration curve for a fluorescence polarization immunoassay method that uses an antibody that has bindability to a target substance and a fluorescently labeled substance in which the target substance is labeled with a fluorescent dye, the method including:
a step 1a of preparing a first reference sample that does not contain the target substance;
a step 1b, after the step 1a, of adding the antibody and the fluorescently labeled substance to the first reference sample and measuring a degree of fluorescence polarization P₀ of the first reference sample;
a step 1c of separating, from a sample to be measured α that contains the target substance at a concentration C_{Sα}, samples to be measured α1 to αp in which the number of samples is an integer p of 3 or greater, adding different amounts of the target substance to each of the samples to be measured α1 to αp, and preparing p calibration curve creation samples 1 to p including a calibration curve creation sample_{conc} in which a concentration of the target substance added later exceeds the original concentration C_{Sα} of the target substance;
a step 1d, after the step 1c, of adding the antibody and the fluorescently labeled substance, in amounts equal to the added amounts in step 1b, to each of the calibration curve creation samples 1 to p, and measuring degrees of fluorescence polarization P₁ to P_{P} of the respective calibration curve creation samples 1 to p; and
a step 1e, after the step 1b and the step 1d, of creating, based on the degree of fluorescence polarization P₀ and a degree of fluorescence polarization P_{conc} of the calibration curve creation sample_{conc} among the calibration curve creation samples 1 to p, a calibration curve expressing a relationship between an amount of the target substance and the degree of fluorescence polarization of the sample.

Step 1a is a step of preparing the first reference sample that does not contain the target substance.

The low-concentration region of the calibration curve created in the present disclosure is created primarily on the basis the degree of fluorescence polarization P₀ of the first reference sample.

Examples of the first reference sample include pure water, a solution that does not contain the target substance from the start, such as physiological saline, and a solution obtained by removing the target substance from the sample to be measured α.

Examples of a method for removing the target substance in the sample to be measured α include a method of adding an enzyme that uses the target substance as a substrate to the sample to be measured α, a method of bringing an adsorbent that adsorbs the target substance into contact with the sample to be measured α, and the like.

When the target substance is histamine, histamine N-methyltransferase, diamine oxidase, and the like can be used as the enzyme. When the target substance is chloramphenicol, chloramphenicol acetyltransferase, hydrolases, nitroreductases, and the like can be used as the enzyme. When the target substance is deoxynivalenol, a mycotoxin-degrading enzyme can be used as the enzyme.

Note that, when using, as the first reference sample, a solution obtained by removing the target substance by adding the enzyme to the sample to be measured α, that first reference sample may include an autofluorescent substance. Accordingly, it is preferable that a corrected degree of fluorescence polarization calculated by Equation (II) above is used as the degree of fluorescence polarization in such cases.

Bentonite, activated carbon, diatomaceous earth, ion exchange adsorbents, affinity adsorbents, and the like can be used as the adsorbent.

Ion exchange adsorbents are adsorbents that contain ion exchange resin or the like as an adsorbing component. Examples of the ion exchange adsorbents include cation exchange adsorbents that adsorb cationic compounds, and anion exchange adsorbents that adsorb anionic compounds. When using an ion exchange adsorbent as the adsorbent, it is possible to efficiently remove the target substance from the sample to be measured α by selecting an appropriate ion exchange adsorbent in accordance with the properties of the target substance, and further adjusting the pH of the sample to be measured α and the pH of the washing fluid.

Affinity adsorbents are adsorbents that utilize enzyme/substrate, antibody/antigen, hormone/receptor protein, and similar biospecific interactions. Affinity adsorbents are particularly preferable as a means for removing the target substance from the sample to be measured α because such use enables the selective removal of the target substance and results in a lower likelihood of adversely affecting the sample to be measured α compared to when using enzyme treatment.

Note that the use method of the adsorbent is not particularly limited. For example, it is possible to remove the target substance from the sample to be measured α by filling a column with the adsorbent and passing the sample to be measured α through the column, or introducing the adsorbent into the sample to be measured α and then filtering out the adsorbent.

Step 1b is a step, after step 1a, of adding the antibody and the fluorescently labeled substance to the first reference sample and measuring the degree of fluorescence polarization P₀ of the first reference sample. It is possible to measure the degree of fluorescence polarization using the fluorescence polarization immunoassay device 1, for example.

As described above, the first reference sample does not contain the target substance. However, provided that the results are not affected, the degree of fluorescence polarization may be measured using a solution obtained by adding a small amount of the target substance to the first reference sample, instead of using the first reference sample. That is, the “degree of fluorescence polarization P₀ of the first reference sample” of the present disclosure includes the “degree of fluorescence polarization of a solution obtained by adding a small amount of the target substance to the first reference sample.”

As described later, the calibration curve of the antigen-antibody reaction in the present disclosure is created as an inverse sigmoid curve as illustrated in FIG. 3, for example. As such, when the antigen concentration (concentration of the target substance) is lower than a certain concentration, the degree of fluorescence polarization hardly changes. In the present disclosure, provided that the concentration is in a range in which there are no significant changes to the degree of fluorescence polarization, it is possible to use, instead of the “first reference sample”, the “solution obtained by adding a small amount of the target substance to the first reference sample”, or a “second reference sample”, described later, in which the amount of the target substance in the solution is a small amount.

Likewise, when the first reference sample is a solution obtained by removing the target substance from the sample to be measured α, provided that the concentration is in a range in which there are no significant changes to the degree of fluorescence polarization, the first reference sample may be a solution that contains a small amount of the target substance that could not be completely removed.

Step 1c is a step of preparing p calibration curve creation samples 1 to p. The calibration curve creation samples 1 to p are obtained by separating, from the sample to be measured α, samples to be measured α1 to αp for which the number of samples is p, and adding different amounts of the target substance to each of the samples to be measured al to ap.

The number (p) of the calibration curve creation samples 1 to p is an integer of 3 or greater and, for example, is an integer of 3 to 20, an integer of 3 to 15, or an integer of 3 to 10.

The calibration curve creation samples 1 to p include the calibration curve creation sample_{conc} in which the concentration of the target substance added later exceeds the original concentration C_{Sα} of the target substance. The p calibration curve creation samples 1 to p may or may not include a calibration curve creation sample_{dil} in which the concentration of the target substance added later is less than or equal to the original concentration C_{Sα} of the target substance.

The medium-concentration to high-concentration region of the calibration curve created in the present disclosure is created primarily on the basis of the degree of fluorescence polarization P_{conc} of the calibration curve creation sample_{conc}.

Specifically, the calibration curve creation samples 1 to p are prepared on the basis of the sample to be measured α and, as such, by using the calibration curve creation samples 1 to p, it is possible to create a calibration curve that takes into account the effects of contaminants in the sample to be measured α.

However, the sample to be measured α originally contains the concentration C_{Sα} of the target substance and, as such, of the calibration curve creation samples 1 to p, calibration curve creation samples in which the concentration of the target substance added later is low (that is, the calibration curve creation sample_{dil}) are not suitable for deriving the relationship between the amount of the target substance added later and the degree of fluorescence polarization of the sample.

Meanwhile, when the amount of the target substance added later is greater than the amount of the target substance originally contained in the sample to be measured α, the target substance originally contained in the sample to be measured α does not hardly exert any effects when deriving the relationship between the amount of the target substance added later and the degree of fluorescence polarization of the sample.

Accordingly, in the present disclosure, among the degrees of fluorescence polarization P₁ to P_{P} of the calibration curve creation samples 1 to p, only the degree of fluorescence polarization P_{conc} of the calibration curve creation sample_{conc} is used.

As described above, in the creation method for a calibration curve of the present disclosure, the calibration curve creation sample_{conc} is a required sample, and the calibration curve creation sample_{dil} is an optional sample.

The number of the calibration curve creation sample_{conc} is, for example, an integer of 2 to 20, an integer of 2 to 15, or an integer of 2 to 10.

The number of the calibration curve creation sample_{dil} is, for example, an integer of 0 to 18, an integer of 0 to 13, or an integer of 0 to 8.

When there is no information related to the concentration C_{Sα} of the target substance when performing step 1c, in step 1c, it is preferable to prepare a wide range of calibration curve creation samples 1 to p from low concentration to high concentration. As a result, in step 1c, the calibration curve creation sample_{dil} is prepared together with the calibration curve creation sample_{conc}.

In this case, the concentration of the most concentrated calibration curve creation sample is, for example, from 10⁶ to 10¹⁰ times the concentration of the least concentrated calibration curve creation sample.

As described later, the calibration curve is typically created as an inverse sigmoid curve by regressing experiment data with a four-parameter logistic model. In order to create a more accurate calibration curve, for example, in the calibration curve illustrated in FIG. 3, it is preferable that a sample having a concentration expressed near degree of polarization d, a sample having a concentration expressed near degree of polarization (a+b)/2, and a sample having a concentration expressed near degree of polarization a are used at the time of creation.

Examples of “near degree of polarization d”, “near degree of polarization (a+b)/2”, and “near degree of polarization a” are specifically respectively d±[(a−d)/5], [(a+b)/2]±[(a−d)×3/5], and a±[(a−d)/5], and are preferably d±[(a−d)/10], [(a+b)/2]+[(a−d)/5], and a+[(a−d)/10].

Meanwhile when there is information related to the concentration C_{Sα} of the target substance when performing step 1c, it is possible to prepare only the calibration curve creation sample_{conc} on the basis of that information. Examples of cases in which there is information related to the concentration C_{Sα} of the target substance include cases in which there is known information about the sample to be measured α, as when analyzing to confirm the components of a food, a beverage, or the like manufactured by a conventional method, and cases in which a provisional concentration C_{Pα} of the target substance in the sample to be measured α is calculated by an additional measurement, described later.

Step 1d is a step, after the step 1c, of adding the antibody and the fluorescently labeled substance to each of the calibration curve creation samples 1 to p, and measuring degrees of fluorescence polarization P₁ to P_{P} of the respective calibration curve creation samples 1 to p.

The added amounts of the antibody and the fluorescently labeled substance are the same as the amounts of the antibody and the fluorescently labeled substance added to the first reference sample in step 1b.

Step 1e is a step, after the step 1b and step 1d, of creating, based on the degree of fluorescence polarization P₀ of the first reference sample and the degree of fluorescence polarization P_{cone} of the calibration curve creation sample_{conc} among the calibration curve creation samples 1 to p, a calibration curve expressing the relationship between the amount of the target substance and the degree of fluorescence polarization of the sample.

Step 1e can, for example, be performed by the following method.

Firstly, some of the calibration curve creation samples are selected, in order from those having high concentration, from the calibration curve creation samples 1 to p, and a calibration curve is created on the basis of the degrees of fluorescence polarization thereof and the degree of fluorescence polarization P₀ of the first reference sample. Next, the degree of fluorescence polarization of the sample to be measured α is measured and the concentration C_{Sα} of the target substance is derived from the calibration curve. By comparing the concentration C_{Sα} of the target substance and the concentration of each calibration curve creation sample, it is possible to determine whether the calibration curve creation sample_{dil} is included in the used calibration curve creation samples.

When the calibration curve creation sample_{dil} is included in the used calibration curve creation samples, the desired calibration curve can be created by repeatedly performing the process of creating a calibration curve while excluding a low-concentration sample from the samples used for creating the calibration curve.

Meanwhile, when the calibration curve creation sample_{dil} is not included in the used calibration curve creation samples, some calibration curve creation samples from among the remaining calibration curve creation samples are added in order from those having high concentration, and the calibration curve is newly created.

As described in the following, if information related to the concentration C_{Sα} of the target substance can be acquired before the creation of the calibration curve, or during the creation of the calibration curve, the calibration curve can be created more efficiently.

For example, in a case in which the calibration curve creation sample_{dil} is prepared together with the calibration curve creation sample_{conc} in step 1c, and the degrees of fluorescence polarization of these are measured in step 1d, by selecting only the degree of fluorescence polarization_{conc} of the calibration curve creation sample_{conc} on the basis of the information related to the concentration C_{Sα} of the target substance acquired after step 1c, it is possible to create the calibration curve on the basis of the degree of fluorescence polarization P_{conc} and the degree of fluorescence polarization P₀ obtained in step 1b.

Examples of the information related to the concentration C_{Sα} of the target substance used at this time include the known information about the sample to be measured α described above, and the provisional concentration C_{Pα} of the target substance in the sample to be measured α calculated by the additional measurement described later.

Meanwhile, in a case in which only the calibration curve creation sample_{conc} is prepared in step 1c, it is possible to create the calibration curve on the basis of the degree of fluorescence polarization P₀ obtained in step 1b and the degrees of fluorescence polarization P₁ to P_{P} obtained in step 1d (that is, the degree of fluorescence polarization P_{conc}).

The “amount of the target substance” in the “relationship between the amount of the target substance and the degree of fluorescence polarization of the sample” of the calibration curve can be appropriately selected in accordance with the use purpose of the calibration curve. Typically, the concentration of the target substance added later in the calibration curve creation samples 1 to p is used as the “amount of the target substance.”

The calibration curve is typically created as an inverse sigmoid curve by regressing experiment data with a four-parameter logistic model.

Note that the aforementioned matters related to the “relationship between the amount of the target substance and the degree of fluorescence polarization of the sample” and the creation method for a calibration curve (for example, the preferable concentration of the sample illustrated in FIG. 3, the method of regressing the experiment data with a four-parameter logistic model, creating the calibration curve as an inverse sigmoid curve, and the like) can be used when deriving a first approximate curve and a second approximate curve, described later.

### Embodiment 1

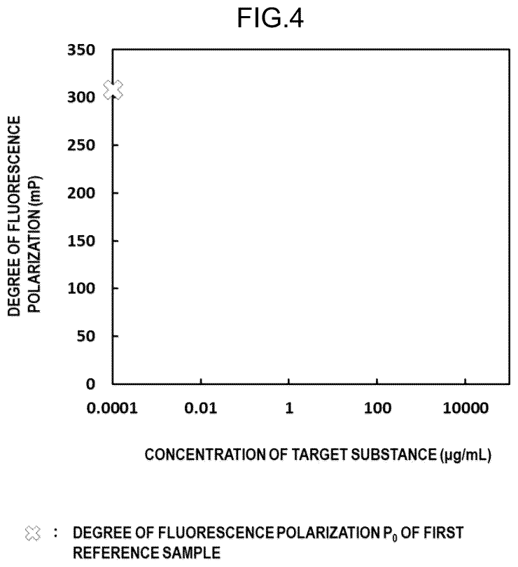

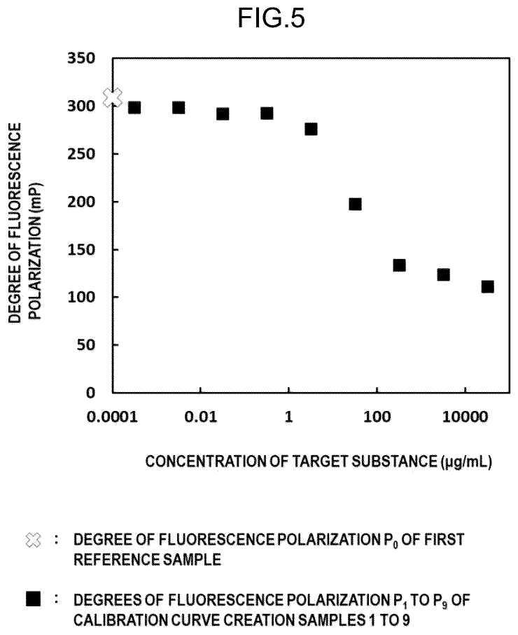

FIGS. 4 to 6 illustrate the processes whereby the calibration curve is created by steps 1a to 1e. However, the creation method for a calibration curve of the present disclosure is not limited to the order of the steps illustrated in FIGS. 4 to 6 and, provided that execution is possible, the order of the steps may be changed.

The creation method for a calibration curve illustrated in FIGS. 4 to 6 is a method in which only the degrees of fluorescence polarization P_{conc} of the plurality of calibration curve creation samples_{conc} is selected in step 1e to create the calibration curve.

FIG. 4 illustrates a state after steps 1a and 1b. The degree of fluorescence polarization P₀ of the first reference sample is plotted on the y axis.

FIG. 5 illustrates a state after steps 1c and 1d. The degrees of fluorescence polarization P₁ to P₉ of a wide range of calibration curve creation samples 1 to 9 from low concentration to high concentration prepared in step 1c in a state in which the concentration C_{Sα} of the target substance is unknown, are plotted.

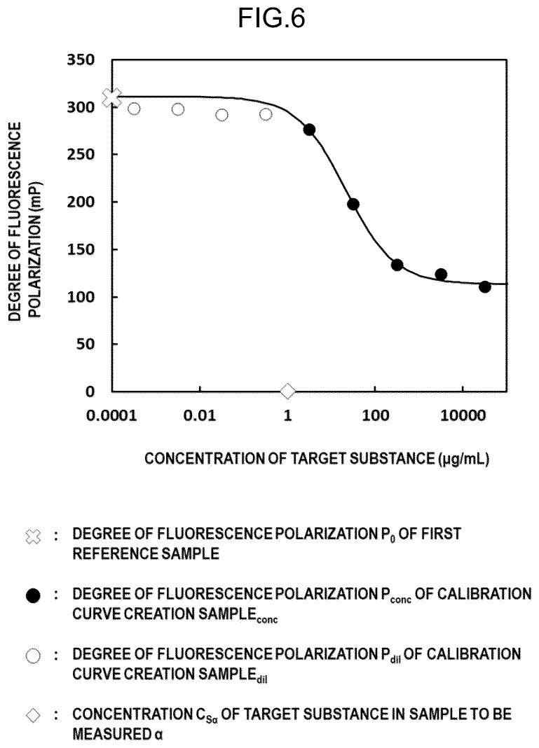

FIG. 6 illustrates a state after step 1e. A calibration curve expressing the relationship between the concentration of the target substance in the sample and the degree of fluorescence polarization of the sample is created on the basis of the degree of fluorescence polarization P₀ of the first reference sample and the degrees of fluorescence polarization P_{conc} of the calibration curve creation samples_{conc} selected on the basis of the information related to the concentration C_{Sα} of the target substance.

Examples of the information related to the concentration C_{Sα} of the target substance used in step 1e include a provisional concentration C_{Pα(1)} of the target substance in the sample to be measured α derived by the following additional measurement 1 (hereinafter, may be abbreviated as “the provisional concentration C_{Pα(1)} of the target substance) and a provisional concentration C_{Pα(2)} of the target substance in the sample to be measured α derived by the following additional measurement 2 (hereinafter, may be abbreviated as “the provisional concentration C_{Pα(2)} of the target substance).

Note that, for convenience of explanation, the tasks that are redundant with steps 1a to 1e are described again but, in actual measurement, the tasks that have already been performed can be omitted in some cases.

### Additional Measurement 1

A Step 2a of Preparing a Second Reference Sample that does not Contain the Target Substance;
A step 2b, after the step 2a, of separating, from the second reference sample, second reference samples 1 to q in which the number of samples is an integer of 3 or greater, adding different amounts of the target substance to each of the second reference samples 1 to q, and preparing q additional measurement samples A1 to Aq;
A step 2c, after the step 2b, of adding the antibody and the fluorescently labeled substance to each of the additional measurement samples A1 to Aq, and measuring degrees of fluorescence polarization P_{A1} to P_{Aq} of the respective additional measurement samples A1 to Aq;
A step 2d, after the step 2c, of deriving, on the basis of the degrees of fluorescence polarization P_{A1} to P_{Aq}, a first approximate curve expressing the relationship between the amount of the target substance and the degree of fluorescence polarization of the sample;
A step 2e of adding the antibody and the fluorescently labeled substance in amounts equal to the added amounts in step 2c to the sample to be measured α, and measuring a degree of fluorescence polarization P_{Xα} of the sample to be measured α; and
A step 2f, after the step 2d and the step 2e, of deriving a provisional concentration C_{Pα(1)} of the target substance in the sample to be measured α from the first approximate curve on the basis of the degree of fluorescence polarization P_{Xα}.

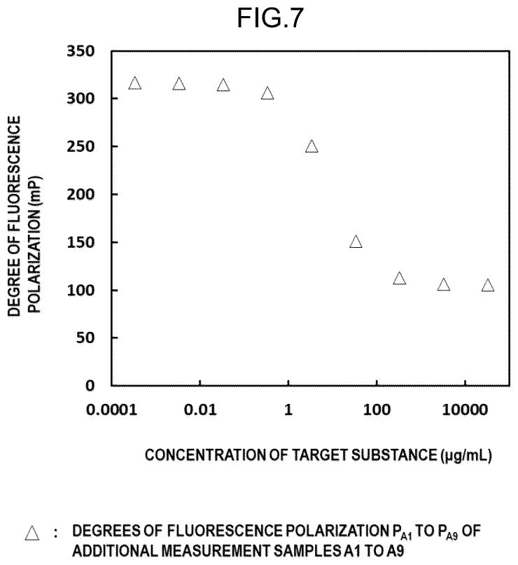

In the following, the processes whereby the first approximate curve and the provisional concentration C_{Pα(1)} of the target substance are derived by steps 2a to 2f are described while referencing FIGS. 7 to 9. However, the creation method for a calibration curve of the present disclosure is not limited to the order of the steps illustrated in FIGS. 7 to 9 and, provided that execution is possible, the order of the steps may be changed.

Step 2a is a step of preparing the second reference sample that does not contain the target substance, and is a step similar to step 1a. However, the second reference sample is used in the preparation of the additional measurement samples A1 to Aq and, as such, in cases in which the target substance is removed by adding the enzyme, the enzyme activity of that enzyme must be inactivated. Accordingly, it is preferable to use, as the second reference sample, a solution that does not contain the target substance from the start, such as pure water or the like, or a solution obtained by removing the target substance from the sample to be measured α using the adsorbent. Note that, as with the first reference sample, when the second reference sample is a solution obtained by removing the target substance from the sample to be measured α, provided that the concentration is in a range in which there are no significant changes to the degree of fluorescence polarization, the second reference sample may be a solution that contains a small amount of the target substance that could not be completely removed.

Step 2b is a step of preparing the q additional measurement samples A1 to Aq. The additional measurement samples A1 to Aq are obtained by separating, from the second reference sample, the second reference samples 1 to q for which the number of samples is q, and adding different amounts of the target substance to these second reference samples 1 to q.

The number (q) of the additional measurement samples A1 to Aq is an integer of 3 or greater and, for example, is an integer of 3 to 20, an integer of 3 to 15, or an integer of 3 to 10. In step 2b, it is preferable to prepare a wide range of additional measurement samples A1 to Aq from low concentration to high concentration. The preferable sample concentration range is as described for the calibration curve creation samples 1 to p.

Step 2c is a step of adding the antibody and the fluorescently labeled substance to each of the additional measurement samples A1 to Aq, and measuring the degrees of fluorescence polarization P_{A1} to P_{Aq} of the respective additional measurement samples A1 to Aq. The added amounts of the antibody and the fluorescently labeled substance may be the same as or different from the amounts of the antibody and the fluorescently labeled substance added to the first reference sample in step 1b.

When the added amounts of the antibody and the fluorescently labeled substance in step 2c are the same as the added amounts in step 1b, redundant measurements can be omitted in some cases. When the added amounts of the antibody and the fluorescently labeled substance in step 2c are different from the added amounts in step 1b, a suitable first approximate curve can be derived for deriving the provisional concentration C_{Pα(1)} of the target substance in some cases.

FIG. 7 illustrates a state after steps 2a to 2c. The degrees of fluorescence polarization P_{A1} to P_{A9} of the additional measurement samples A1 to A9 are plotted.

Step 2d is a step, after the step 2c, of deriving, on the basis of the degrees of fluorescence polarization P_{A1} to P_{Aq}, the first approximate curve expressing the relationship between the amount of the target substance and the degree of fluorescence polarization of the sample.

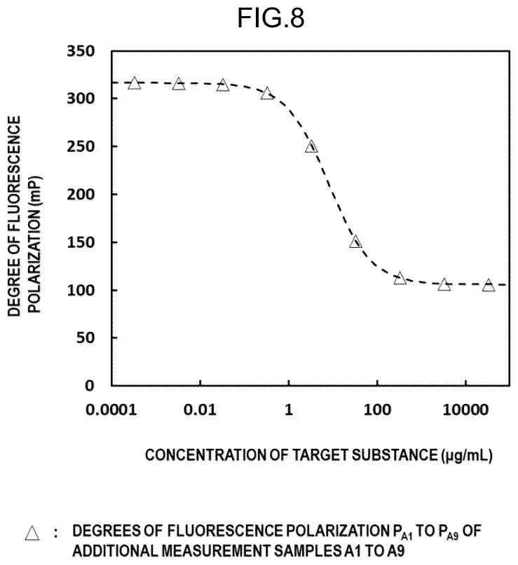

FIG. 8 illustrates a state after step 2d. The degrees of fluorescence polarization P_{A1} to P_{A9} of the additional measurement samples A1 to A9 and the first approximate curve derived on the basis of the degrees of fluorescence polarization P_{A1} to P_{A9} are plotted.

Step 2e is a step of adding the antibody and the fluorescently labeled substance to the sample to be measured α and measuring the degree of fluorescence polarization P_{Xα} of the sample to be measured α. The added amounts of the antibody and the fluorescently labeled substance are the same as the amounts of the antibody and the fluorescently labeled substance added A1 to the additional measurement samples A1 to Aq in step 2c.

Step 2f is a step of deriving the provisional concentration C_{Pα(1)} of the target substance from the first approximate curve on the basis of the degree of fluorescence polarization P_{Xα} of the sample to be measured α.

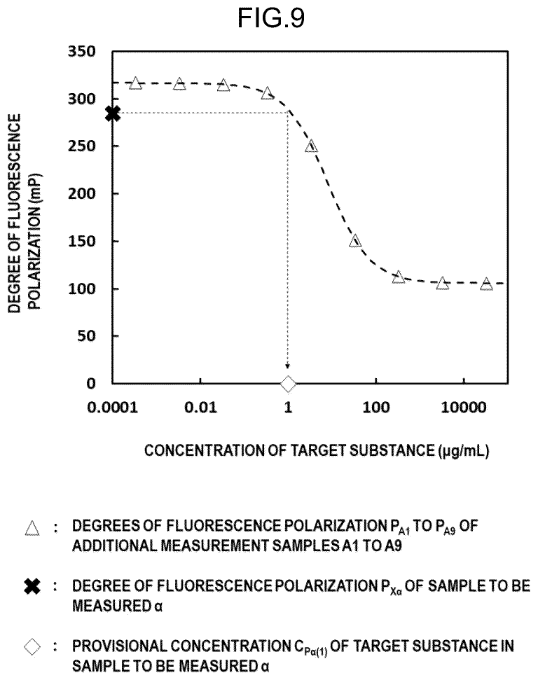

FIG. 9 illustrates a state after steps 2e and 2f. The degree of fluorescence polarization P_{Xα} of the sample to be measured α is plotted on the y axis, and the provisional concentration C_{Pα(1)} of the target substance corresponding thereto is plotted on the x axis.

### Additional Measurement 2

A step 3a of preparing the second reference sample that does not contain the target substance;
A step 3b, after the step 3a, of separating, from the second reference sample, second reference samples 1 to q in which the number of samples is an integer q of 3 or greater, adding different amounts of the target substance to each of the second reference samples 1 to q, and preparing q additional measurement samples A1 to Aq;
A step 3c, after the step 3b, of adding the antibody and the fluorescently labeled substance to each of the additional measurement samples A1 to Aq, and measuring degrees of fluorescence polarization P_{A1} to P_{Aq} of the respective additional measurement samples A1 to Aq;
A step 3d, after the step 3c, of deriving, on the basis of the degrees of fluorescence polarization P_{A1} to P_{Aq}, a first approximate curve expressing the relationship between the amount of the target substance and the degree of fluorescence polarization of the sample;
A step 3e of separating, from the sample to be measured α, samples to be measured α1 to αr in which the number of samples is an integer r of 3 or greater, adding different amounts of the target substance to each of the samples to be measured α1 to αr, and preparing r additional measurement samples B1 to Br;
A step 3f, after the step 3e, of adding the antibody and the fluorescently labeled substance, in amounts equal to the added amounts in step 3c, to each of the additional measurement samples B1 to Br, and measuring degrees of fluorescence polarization P_{B1} to P_{Br} of the respective additional measurement samples B1 to Br;
A step 3g, after the step 3f, of deriving, based on the degrees of fluorescence polarization P_{B1} to P_{Br}, a second approximate curve expressing the relationship between the amount of the target substance and the degree of fluorescence polarization of the sample; and
A step 3h, after the step 3d and the step 3g, of deriving, from the first approximate curve and based on a maximum value of the degrees of fluorescence polarization on the second approximate curve, a provisional concentration C_{Pα(2)} of the target substance in the sample to be measured α.

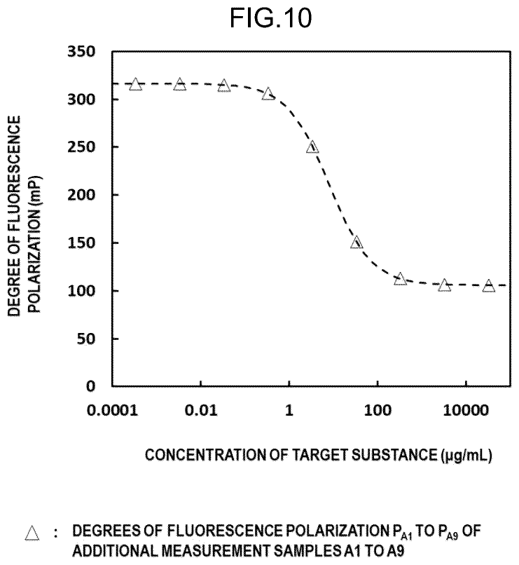

In the following, the processes whereby the first approximate curve, the second approximate curve, and the provisional concentration C_{Pα(2)} of the target substance are derived by steps 3a to 3h are described while referencing FIGS. 10 to 12. However, the creation method for a calibration curve of the present disclosure is not limited to the order of the steps illustrated in FIGS. 10 to 12 and, provided that execution is possible, the order of the steps may be changed.

In the steps 3a to 3d, the same tasks as in steps 2a to 2d are performed.

FIG. 10 illustrates a state after steps 3a to 3d. The degrees of fluorescence polarization P_{A1} to P_{A9} of the additional measurement samples A1 to A9 and the first approximate curve derived on the basis of the degrees of fluorescence polarization P_{A1} to P_{A9} are plotted.

Step 3e is a step of preparing the r additional measurement samples B1 to Br. Step 3f is a step or measuring degrees of the fluorescence polarization P_{B1} to P_{Br} of each of the additional measurement samples B1 to Br. As with the calibration curve creation samples 1 to p, the additional measurement samples B1 to Br are prepared from the sample to be measured α. Accordingly, the steps 3e and 3f include the same tasks as in steps 1c and 1d, namely preparing the calibration curve creation samples 1 to p in a state in which there is no information related to the concentration C_{Sα} of the target substance, and measuring the degrees of fluorescence polarization P₁ to Pₚ of the calibration curve creation samples 1 to p. As such, in step 3c, when using the antibody and the fluorescently labeled substance in amounts equal to the added amounts in step 1b, the measurement results of step 1d can be used as the measurement results of step 3f in some cases.

In step 3g, the second approximate curve expressing the relationship between the amount of the target substance and the degree of fluorescence polarization is derived on the basis of the degrees of fluorescence polarization P_{B1} to P_{Br} of the additional measurement samples B1 to Br.

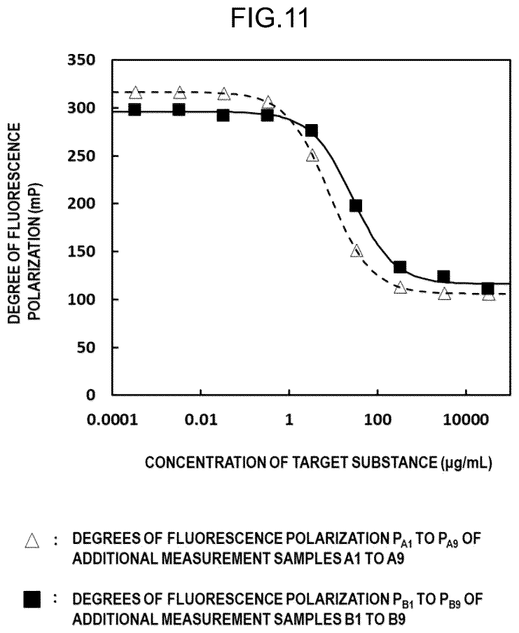

FIG. 11 illustrates a state after steps 3e to 3g. The degrees of fluorescence polarization P_{B1} to P_{B9} of the additional measurement samples B1 to B9 and the second approximate curve derived on the basis of the degrees of fluorescence polarization P_{B1} to P_{B9} are plotted.

Step 3h is a step of deriving the provisional concentration C_{Pα(2)} of the target substance from the first approximate curve on the basis of the maximum value of the degrees of fluorescence polarization on the second approximate curve.

The degree of fluorescence polarization when the concentration of the added target substance is 0 in the second approximate curve represents the degree of fluorescence polarization of the sample to be measured α. Accordingly, the maximum value of the degrees of fluorescence polarization on the second approximate curve (the value of the intersection of the second approximate curve and the y axis) is read, and the provisional concentration C_{Pα(2)} of the target substance can be derived from the first approximate curve on the basis of the maximum value.

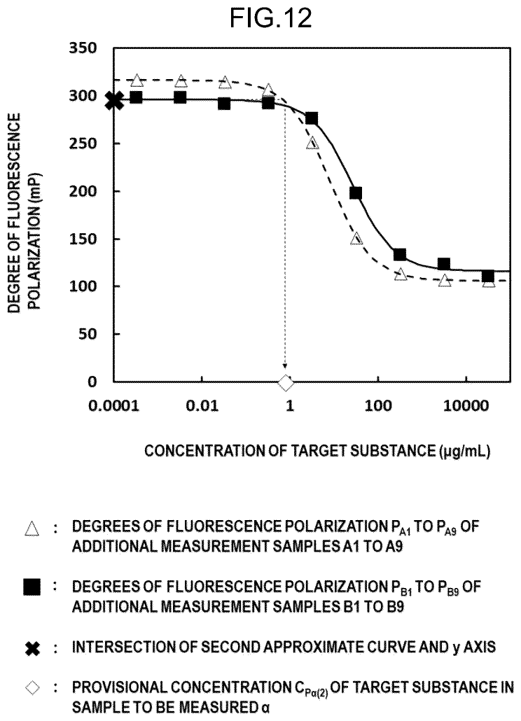

FIG. 12 illustrates a state after step 3 h. The intersection of the second approximate curve and the y axis is plotted, and the provisional concentration C_{Pα(2)} of the target substance corresponding thereto is plotted on the x axis.

### OTHER EMBODIMENTS

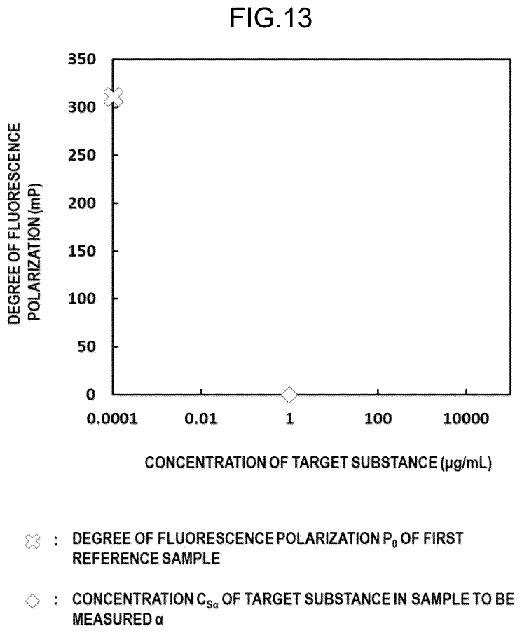

When there is information related to the concentration C_{Sα} of the target substance when performing step 1c, it is possible to create the calibration curve by the further simplified method illustrated in FIGS. 13 to 15.

The creation method for a calibration curve illustrated in FIGS. 13 to 15 is a method in which only the plurality of calibration curve creation samples_{conc} is prepared in step 1c.

FIG. 13 illustrates a state after steps 1a and 1b. The degree of fluorescence polarization P₀ of the first reference sample is plotted on the y axis. Additionally, there is information, as known information about the sample to be measured α, that the concentration C_{Sα} of the target substance is about 1 μg/mL, and this is plotted on the x axis.

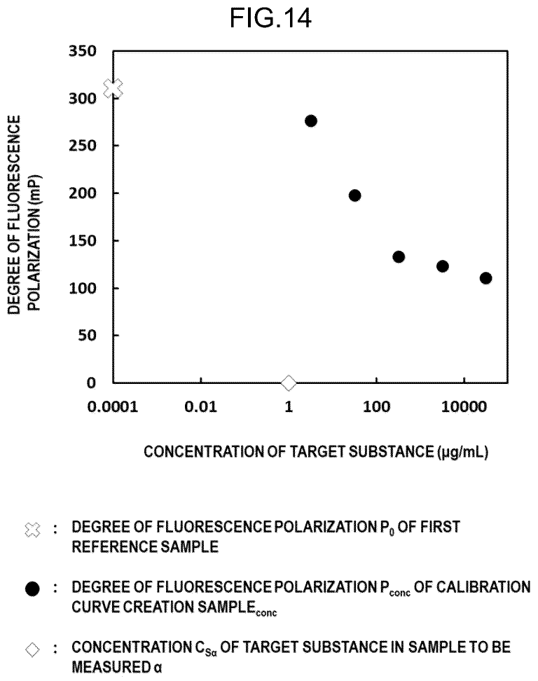

FIG. 14 illustrates a state after steps 1c and 1d. The degrees of fluorescence polarization P_{conc} of the calibration curve creation samples_{conc} prepared on the basis of the information related to the concentration C_{Sα} of the target substance are plotted.

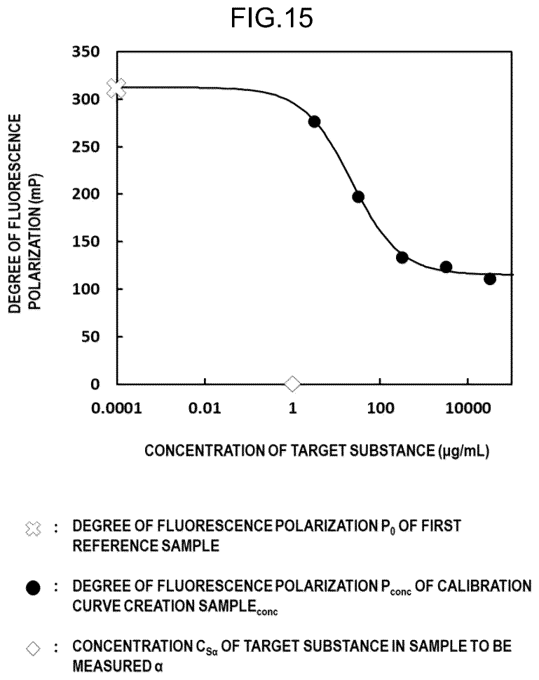

FIG. 15 illustrates a state after step 1e. A calibration curve expressing the relationship between the concentration of the target substance in the sample and the degree of fluorescence polarization is created on the basis of the degree of fluorescence polarization P₀ of the first reference sample and the degrees of fluorescence polarization P_{cone} of the calibration curve creation samples_{conc}.

The calibration curve created by the present disclosure as described above is created by combining a low-concentration region sample (the first reference sample) and medium-concentration to high-concentration region samples (the calibration curve creation samples_{conc}), and is more accurate in a wide range from the low-concentration region to the high-concentration region.

### 2. Fluorescence Polarization Immunoassay Method

A fluorescence polarization immunoassay method according to a second aspect of the present disclosure uses the antibody, that has bindability to the target substance, and the fluorescently labeled substance, in which the target substance is labeled with a fluorescent dye, to measure a concentration C_{Sβ} of the target substance in a sample to be measured β, the method including:
a step 4a of adding the antibody and the fluorescently labeled substance in amounts equal to the added amounts in the step 1b to the sample to be measured β, and measuring a degree of fluorescence polarization P_{Xβ} of the sample to be measured β; and
a step 4b, after the step 4a, of deriving, based on the degree of fluorescence polarization P_{Xβ}, the concentration C_{Sβ} of the target substance from the calibration curve created by the method.

That is, the fluorescence polarization immunoassay method of the present disclosure includes measuring the degree of fluorescence polarization P_{Xβ} of the sample to be measured β, and measuring the concentration C_{Sβ} of the target substance from the calibration curve created by the method.

A sample that is the same as the sample to be measured α, or a sample that is of the same type as the sample to be measured α is preferably used as the sample to be measured β.

Examples of cases in which the sample to be measured β is the same as the sample to be measured α include when creating the calibration curve by the method described above and, then, using the remainder of the sample to be measured α used to create the calibration curve to measure the degree of fluorescence polarization and derive the concentration thereof. In step 1c and step 1e, when performing the additional measurement 1 and, at that time, using the antibody and the fluorescently labeled substance in amounts equal to the added amounts in step 1b to measure the degree of fluorescence polarization P_{Xα} of the sample to be measured α, step 4a is already performed and, as such, the concentration C_{Sα} of the target substance is derived simultaneously with the completion of the calibration curve.

Examples of cases in which the sample to be measured β is the same type as the sample to be measured α include when analyzing the components of a product of another lot manufactured by the same method or a similar method at a factory or the like.

### 3. Program

A program according to a third aspect of the present disclosure is used in the fluorescence polarization immunoassay method according to the second aspect of the present disclosure, and causes a computer to execute a step of deriving, based on the degree of fluorescence polarization P_{Xβ} of the sample to be measured β, the concentration C_{Sβ} of the target substance from the calibration curve created by the method.

Each step of the program according to the third aspect of the present disclosure is executed by a computer. In one example, the computer includes a processor, a memory, and an input/output device. The processor is configured from a central processing unit (CPU), or the like, and executes a program stored in the memory. The memory is an example of a storage medium, and stores the program according to the third aspect and the calibration curve obtained by the creation method for a calibration curve according to the first aspect. This program causes the processor to execute calculations of the fluorescence polarization immunoassay method according to the second aspect. The input/output device inputs the degree of fluorescence polarization P_{Xβ} required for the calculations of the fluorescence polarization immunoassay method according to the second aspect. Additionally, the input/output device outputs the results of the calculations, that is, displays the concentration C_{Sβ}, or the like. The processor performs, in accordance with the program according to the third aspect, the calculations of the fluorescence polarization immunoassay method according to the second aspect on the degree of fluorescence polarization P_{Xβ} input by the input/output device, derives the concentration C_{Sβ}, and outputs the derived concentration C_{Sβ} from the input/output device.

The program of the present disclosure is preferably used when the sample to be measured β is of the same type as the sample to be measured β.

Specifically, by initially creating an accurate calibration curve when analyzing the components of a product manufactured at a factory or the like, it is, thereafter, possible to use that calibration curve when analyzing the components of a product of another lot manufactured by the same method or a similar method. As such, it is possible to efficiently analyze the components of a product by using the program of the present disclosure.

### 4. Fluorescence Polarization Immunoassay Device

A fluorescence polarization immunoassay device according to a fourth aspect of the present disclosure uses an antibody, that has bindability to a target substance, and a fluorescently labeled substance, in which the target substance is labeled with a fluorescent dye, to measure a concentration C_{Sβ} of the target substance in a sample to be measured β, the device including:
an emission optical system that emits linearly polarized excitation light on a sample;
a polarized light adjustment element that selectively allows, of fluorescence emitted from the sample, a linearly polarized component corresponding to a drive signal to pass through;
a receiver that detects fluorescence intensity that has passed through the polarized light adjustment element; and
a controller that outputs the drive signal to the polarized light adjustment element and measures, in accordance with the drive signal and based on the fluorescence intensity detected by the receiver, a degree of polarization of the sample, wherein
the controller includes a storage medium that stores the calibration curve created by the method according to the first aspect and a program used in the fluorescence polarization immunoassay method according to the second aspect, the program causing a computer to execute:
a step of deriving, based on the degree of fluorescence polarization P_{Xβ} of the sample to be measured β, the concentration C_{Sβ} of the target substance from the calibration curve.

The emission optical system, the polarized light adjustment element, and the receiver of the fluorescence polarization immunoassay device of the present disclosure are the same as those of the fluorescence polarization immunoassay device 1 described above.

In one example, the controller of the fluorescence polarization immunoassay device of the present disclosure includes a processor, a memory, and an input/output device. The controller of the fluorescence polarization immunoassay device of the present disclosure includes a storage medium such as the memory, and stores the calibration curve created by the method according to the first aspect and a program used in the fluorescence polarization immunoassay method according to the second aspect. The controller of the fluorescence polarization immunoassay device of the present disclosure can execute each step of the program of the present disclosure and, as such, can efficiently measure the concentration C_{Sβ} of the target substance by using the fluorescence polarization immunoassay device of the present disclosure.

### 5. Calibration Curve Creation Kit

A calibration curve creation kit according to a fifth aspect of the present disclosure includes:
an antibody that has bindability to a target substance, and a fluorescently labeled substance, in which the target substance is labeled with a fluorescent dye; and further includes a means for removing the target substance from a solution containing the target substance, and/or a liquid not containing the target substance.

The antibody that has bindability to the target substance and the fluorescently labeled substance in which the target substance is labeled with a fluorescent dye are described in the disclosure of the creation method for a calibration curve.

These may be in a solution state or in a dry state.

These are typically stored in containers such bags, bottles, or ampoules in accordance with their respective states.

Examples of the means for removing the target substance from the solution containing the target substance include a substance for removing the target substance from the solution containing the target substance, such as enzymes, adsorbents, and the like. These are described in the disclosure of the creation method for a calibration curve.

Examples of the liquid not containing the target substance include pure water, physiological saline, buffer solutions, and the like.

It is preferable that the calibration curve creation kit of the present disclosure includes just the right amount of the antibody and the fluorescently labeled substance in accordance with an anticipated number of uses.

The calibration curve creation kit of the present disclosure may further include the present disclosure microdevice.

The calibration curve creation kit of the present disclosure is preferably used when executing the creation method for a calibration curve of the present disclosure described above.

### EXAMPLES

### Reagent

### (1) Antibody Solution

An anti-histamine antibody (manufactured by Progen Biotechnik) was diluted with phosphate buffered saline (PBS(−)) containing 0.01% bovine serum albumin (BSA) to prepare a 1.3×10⁻⁷ M antibody solution.

### (2) Histamine Tracer Solution

Histamine (manufactured by Fujifilm Wako Pure Chemical Industries, Ltd.) was modified with HiLyte Fluor 647 to obtain a histamine tracer. This histamine tracer was dissolved in pure water and then further diluted with PBS(−) to prepare a 4.56×10⁻⁹ M histamine tracer solution.

### (3) Target Substance Solution

Histamine dihydrochloride (manufactured by Fujifilm Wako Pure Chemical Industries, Ltd.) was dissolved in pure water to prepare a 32 mg/ml histamine solution. This histamine solution was diluted with pure water to prepare histamine solutions (#1 to #9) with the following concentrations.

$$2.88 \times 1 {0}^{- 9} M ( 0.00032 μg / mL ) \qquad \#1$$

$$2.88 \times 1 {0}^{- 8} M ( 0.0032 μg / mL ) \qquad \#2$$

$$2.88 \times 1 {0}^{- 7} M ( 0.032 μg / mL ) \qquad \#3$$

$$2.88 \times 1 {0}^{- 6} M ( 0.32 μg / mL ) \qquad \#4$$

$$2.88 \times 1 {0}^{- 5} M ( 3.2 μg / mL ) \qquad \#5$$

$$0.000288 M ( 32 μg / mL ) \qquad \#6$$

$$0.002879 M ( 320 μg / mL ) \qquad \#7$$

$$0.02879 M ( 3 , 200 μg / mL ) \qquad \#8$$

$$0.287899 M ( 32 , 000 μg / mL ) \qquad \#9$$

### (4) Reference Sample (e)

A histamine-decomposing enzyme was added to soy sauce to prepare a reference sample (e). As a histamine-decomposing enzyme reagent, the reagent from a histamine measurement kit (Check Color Histamine (manufactured by Kikkoman Biochemifa)) was used according to the protocols of that kit.

### (5) Reference Sample (w)

Pure water was used as a reference sample (w).

### (6) Reference Sample (f)

Soy sauce was diluted with a 20 mM phosphate buffer solution having a pH of 6.0 to prepare a soy sauce dilution. This soy sauce dilution was passed through a column (Sep-Pak Plus Accell CM, manufactured by Waters). Next, a washing fluid (20 mM phosphate buffer solution having a pH of 6.0) was passed through the column. A combination of the soy sauce dilution and the washing fluid passed through the column was used as the reference sample (f).

### (7) Acylation Reagent and Acylation Buffer

The reagent of the Histamine Acylation Set (manufactured by Beckman Coulter) was used as the acylation reagent and acylation buffer.

### Example 1

The concentration of the histamine (the target substance) contained in the soy sauce (the sample to be measured) was measured by the following method. Example 1 is an example in which there is no known information related to the histamine concentration in the soy sauce. The calibration curve creation sample_{conc} and the calibration curve creation sample_{dil} were prepared and, the degrees of fluorescence polarization thereof were measured and, then, the calibration curve creation sample_{conc} was selected to create a calibration curve.

Note that the degree of fluorescence polarization in Example 1 is the corrected degree of fluorescence polarization calculated by Equation (II) above.

160 μl of PBS(−) was added to 40 μl of the reference sample (e) to obtain a five-fold diluted solution. 100 μl of the obtained five-fold diluted solution, 25 μl of the acylation reagent, and 200 μl of the acylation buffer were mixed to obtain a mixed liquid. 25 μl of this mixed liquid, 25 μl of the histamine tracer solution, and 25 μl of the antibody solution were mixed, and the mixture was allowed to rest in a light shielded environment for 10 minutes at room temperature to prepare a reference sample (e)-containing measurement liquid, and the degree of fluorescence polarization of the reference sample (e)-containing measurement liquid was measured.

40 μl of soy sauce and 60 μl of PBS(−) were added to 100 μl of each of the histamine solutions #1 to #9 to obtain calibration curve creation samples (#1) to (#9). 100 μl of the calibration curve creation samples (#1) to (#9), 25 μl of the acylation reagent, and 200 μl of the acylation buffer were mixed to obtain mixed liquids (#1) to (#9). 25 μl of the mixed liquids (#1) to (#9), 25 μl of the histamine tracer solution, and 25 μl of the antibody solution were mixed, and these mixtures were allowed to rest in a light shielded environment for 10 minutes at room temperature to prepare calibration curve creation sample-containing measurement liquids (#1) to (#9), and the degrees of fluorescence polarization of these calibration curve creation sample-containing measurement liquids (#1) to (#9) were measured.

40 μl of the reference sample (w) and 60 μl of PBS(−) were added to 100 μl of each of the histamine solutions #1 to #9 to obtain additional measurement samples A (#1) to (#9). 100 μl of the additional measurement samples A (#1) to (#9), 25 μl of the acylation reagent, and 200 μl of the acylation buffer were mixed to obtain mixed liquids (#1) to (#9). 25 μl of the mixed liquids (#1) to (#9), 25 μl of the histamine tracer solution, and 25 μl of the antibody solution were mixed, and these mixtures were allowed to rest in a light shielded environment for 10 minutes at room temperature to prepare additional measurement sample A-containing measurement liquids (#1) to (#9), and the degrees of fluorescence polarization of these additional measurement sample A-containing measurement liquids (#1) to (#9) were measured. Next, the first approximate curve was derived from the obtained degrees of fluorescence polarization.

The degrees of fluorescence polarization of the calibration curve creation samples (#1) to (#9) were used as the degrees of fluorescence polarization of the additional measurement samples B (#1) to (#9) to derive the second approximate curve. The maximum value of the degrees of fluorescence polarization on the second approximate curve was read, and the provisional concentration C_{Pα} of the histamine in the soy sauce was derived from the first approximate curve on the basis of the maximum value.

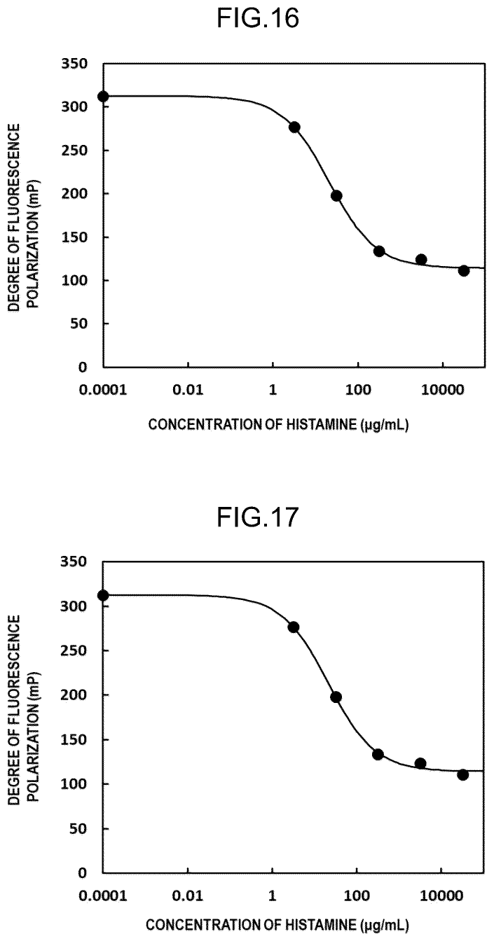

On the basis of the provisional concentration C_{Pα}, the calibration curve creation samples (#1) to (#9) were divided into the calibration curve creation samples_{conc} (calibration curve creation samples (#5) to (#9)) and the calibration curve creation samples_{dil} (calibration curve creation samples (#1) to (#4)), and a calibration curve was created from the degree of fluorescence polarization of the reference sample (e) and the degrees of fluorescence polarization of the calibration curve creation samples (#5) to (#9). The obtained calibration curve is illustrated in FIG. 16.

### Example 2

With the exception of using the reference sample (w) instead of the reference sample (e) in Example 1, and using the degree of fluorescence polarization calculated by Equation (I) described above, in Example 2, the calibration curve was created in the same manner as in Example 1. The obtained calibration curve is illustrated in FIG. 17.

### Example 3

The concentration of the histamine (the target substance) contained in the soy sauce (the sample to be measured) was measured by the following method. Example 3 is an example in which only the calibration curve creation sample_{conc} is prepared on the basis of known information related to the histamine concentration in the soy sauce.

Note that the degree of fluorescence polarization in Example 3 is the corrected degree of fluorescence polarization calculated by Equation (II) above.

160 μl of PBS(−) was added to 40 μl of the reference sample (e) to obtain a five-fold diluted solution. 100 μl of the obtained five-fold diluted solution, 25 μl of the acylation reagent, and 200 μl of the acylation buffer were mixed to obtain a mixed liquid. 25 μl of this mixed liquid, 25 μl of the histamine tracer solution, and 25 μl of the antibody solution were mixed, and the mixture was allowed to rest in a light shielded environment for 10 minutes at room temperature to prepare a reference sample (e)-containing measurement liquid, and the degree of fluorescence polarization of the reference sample (e)-containing measurement liquid was measured.

40 μl of soy sauce and 60 μl of PBS(−) were added to 100 μl of each of the histamine solutions #5 to #9 to obtain calibration curve creation samples_{conc} (#5) to (#9). 100 μl of the calibration curve creation samples_{conc} (#5) to (#9), 25 μl of the acylation reagent, and 200 μl of the acylation buffer were mixed to obtain mixed liquids (#5) to (#9). 25 μl of the mixed liquids (#5) to (#9), 25 μl of the histamine tracer solution, and 25 μl of the antibody solution were mixed, and these mixtures were allowed to rest in a light shielded environment for 10 minutes at room temperature to prepare calibration curve creation sample_{conc}-containing measurement liquids (#5) to (#9), and the degrees of fluorescence polarization of these calibration curve creation sample_{conc}-containing measurement liquids (#1) to (#9) were measured.

A calibration curve similar to that of Example 1 was created from the degree of fluorescence polarization of the reference sample (e) and the degrees of fluorescence polarization of the calibration curve creation samples_{conc} (#5) to (#9).

### Example 4

With the exception of using the reference sample (w) instead of the reference sample (e) in Example 3, and using the degree of fluorescence polarization calculated by Equation (I) described above, in Example 4, a calibration curve similar to that of Example 2 was created in the same manner as in Example 3.

### Example 5

160 μl of PBS(−) was added to 40 μl of the soy sauce to obtain a five-fold diluted solution. 100 μl of the obtained five-fold diluted solution, 25 μl of the acylation reagent, and 200 μl of the acylation buffer were mixed to obtain a mixed liquid. 25 μl of this mixed liquid, 25 μl of the histamine tracer solution, and 25 μl of the antibody solution were mixed, and the mixture was allowed to rest in a light shielded environment for 10 minutes at room temperature to prepare a soy sauce-containing measurement liquid, and the degree of fluorescence polarization of the soy sauce-containing measurement liquid was measured.

When the histamine concentration in the soy sauce was calculated from the calibration curve created in Example 2, the histamine concentration was 1.02 ppm.

Note that, when the histamine concentration in the soy sauce was calculated from the first approximate curve created in the process of Example 1, the histamine concentration was 0.69 ppm.

### Example 6

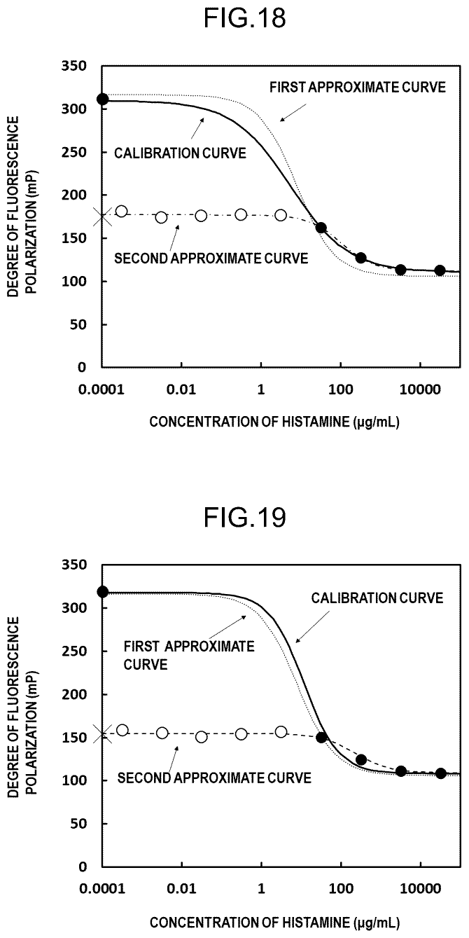

With the exception of using, as the sample to be measured, soy sauce different than the soy sauce used in Examples 1 to 5, in Example 6, the calibration curve was created in the same manner as in Example 1. The obtained calibration curve is illustrated in FIG. 18.

### Example 7

With the exception of using fish sauce as the sample to be measured, in Example 7, the calibration curve was created in the same manner as in Example 1. The obtained calibration curve is illustrated in FIG. 19.

### Example 8

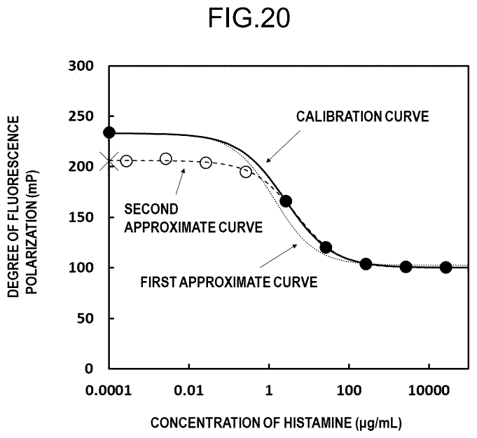

With the exceptions of using the reference sample (f) instead of the reference sample (e) in Example 1, and using the degree of fluorescence polarization calculated by Equation (I) described above, in Example 8, the calibration curve was created in the same manner as in Example 1. The obtained calibration curve is illustrated in FIG. 20.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A method for creating a calibration curve for a fluorescence polarization immunoassay that uses an antibody that has bindability to a target substance and a fluorescently labeled substance in which the target substance is labeled with a fluorescent dye, the method comprising:
a step 1a of preparing a first reference sample that does not contain the target substance;
a step 1b, after the step 1a, of adding the antibody and the fluorescently labeled substance to the first reference sample and measuring a degree of fluorescence polarization P₀ of the first reference sample;
a step 1c of separating, from a sample to be measured β that contains the target substance at a concentration C_{Sα}, samples to be measured al to ap in which the number of samples is an integer p of 3 or greater, adding different amounts of the target substance to each of the samples to be measured al to ap, and preparing p calibration curve creation samples 1 to p including a calibration curve creation sample_{conc} in which a concentration of the target substance added later exceeds the original concentration C_{Sα} of the target substance;
a step 1d, after the step 1c, of adding the antibody and the fluorescently labeled substance, in amounts equal to the added amounts in step 1b, to each of the calibration curve creation samples 1 to p, and measuring degrees of fluorescence polarization P₁ to P_{P} of the respective calibration curve creation samples 1 to p; and
a step 1e, after the step 1b and the step 1d, of creating, based on the degree of fluorescence polarization P₀ and a degree of fluorescence polarization P_{cone} of the calibration curve creation sample_{conc} among the calibration curve creation samples 1 to p, a calibration curve expressing a relationship between an amount of the target substance and the degree of fluorescence polarization of the sample.

2. The method for creating a calibration curve according to claim 1, wherein
in the step 1c, only a plurality of the calibration curve creation sample_{conc} is prepared based on information related to the concentration C_{Sα} of the target substance, or
in the step 1e, only the degree of fluorescence polarization P_{conc} of the plurality of calibration curve creation samples_{conc} is selected based on the information related to the concentration C_{Sα} of the target substance.

3. The method for creating a calibration curve according to claim 2, wherein
the information related to the concentration C_{Sα} of the target substance is a provisional concentration C_{Pα(1)} of the target substance obtained by an additional measurement 1 including:
a step 2a of preparing a second reference sample that does not contain the target substance;
a step 2b, after the step 2a, of separating, from the second reference sample, second reference samples 1 to q in which the number of samples is an integer q of 3 or greater, adding different amounts of the target substance to each of the second reference samples 1 to q, and preparing q additional measurement samples A1 to Aq;
a step 2c, after the step 2b, of adding the antibody and the fluorescently labeled substance to each of the additional measurement samples A1 to Aq, and measuring degrees of fluorescence polarization P_{A1} to P_{Aq} of the respective additional measurement samples A1 to Aq;
a step 2d, after the step 2c, of deriving, based on the degrees of fluorescence polarization P_{A1} to P_{Aq}, a first approximate curve expressing the relationship between the amount of the target substance and the degree of fluorescence polarization of the sample;
a step 2e of adding the antibody and the fluorescently labeled substance, in amounts equal to the added amounts in step 2c, to the sample to be measured β, and measuring a degree of fluorescence polarization P_{Xα} of the sample to be measured β; and
a step 2f, after the step 2d and the step 2e, of deriving, based on the degree of fluorescence polarization P_{Xα}, the provisional concentration C_{Pα(1)} of the target substance in the sample to be measured α from the first approximate curve.

4. The method for creating a calibration curve according to claim 2, wherein
the information related to the concentration C_{Sα} of the target substance is a provisional concentration C_{Pα(2)} of the target substance obtained by an additional measurement 2 including:
a step 3a of preparing a second reference sample that does not contain the target substance;
a step 3b, after the step 3a, of separating, from the second reference sample, second reference samples 1 to q in which the number of samples is an integer q of 3 or greater, adding different amounts of the target substance to each of the second reference samples 1 to q, and preparing q additional measurement samples A1 to Aq;
a step 3c, after the step 3b, of adding the antibody and the fluorescently labeled substance to each of the additional measurement samples A1 to Aq, and measuring degrees of fluorescence polarization P_{A1} to P_{Aq} of the respective additional measurement samples A1 to Aq;
a step 3d, after the step 3c, of deriving, based on the degrees of fluorescence polarization P_{A1} to P_{Aq}, a first approximate curve expressing the relationship between the amount of the target substance and the degree of fluorescence polarization of the sample;
a step 3e of separating, from the sample to be measured α, samples to be measured α1 to αr in which the number of samples is an integer r of 3 or greater, adding different amounts of the target substance to each of the samples to be measured α1 to αr, and preparing r additional measurement samples B1 to Br;
a step 3f, after the step 3e, of adding the antibody and the fluorescently labeled substance, in amounts equal to the added amounts in step 3c, to each of the additional measurement samples B1 to Br, and measuring degrees of fluorescence polarization P_{B1} to P_{Br} of the respective additional measurement samples B1 to Br;
a step 3g, after the step 3f, of deriving, based on the degrees of fluorescence polarization P_{B1} to P_{Br}, a second approximate curve expressing the relationship between the amount of the target substance and the degree of fluorescence polarization of the sample; and
a step 3h, after the step 3d and the step 3g, of deriving, based on a maximum value of the degrees of fluorescence polarization on the second approximate curve, a provisional concentration C_{Pα(2)} of the target substance in the sample to be measured α from the first approximate curve.

5. The method for creating a calibration curve according to claim 2, wherein the information related to the concentration C_{Sα} is known information about the sample to be measured α.

6. The method for creating a calibration curve according to claim 1, wherein the first reference sample is pure water.

7. The method for creating a calibration curve according to claim 1, wherein the first reference sample is a solution obtained by removing the target substance from the sample to be measured α.

8. The method for creating a calibration curve according to claim 7, wherein a method for removing the target substance in the sample to be measured α is a method of adding an enzyme that uses the target substance as a substrate to the sample to be measured α.

9. The method for creating a calibration curve according to claim 7, wherein a method for removing the target substance in the sample to be measured α is a method of bringing an adsorbent that adsorbs the target substance into contact with the sample to be measured α.

10. The method for creating a calibration curve according to claim 3, wherein the second reference sample is pure water.

11. The method for creating a calibration curve according to claim 3, wherein the second reference sample is a solution obtained by removing the target substance from the sample to be measured α.

12. The method for creating a calibration curve according to claim 11, wherein a method for removing the target substance in the sample to be measured α is a method of adding an enzyme that uses the target substance as a substrate to the sample to be measured α.

13. The method for creating a calibration curve according to claim 11, wherein a method for removing the target substance in the sample to be measured α is a method of bringing an adsorbent that adsorbs the target substance into contact with the sample to be measured α.

14. The method for creating a calibration curve according to claim 1, wherein the calibration curve is created by regressing experiment data with a four-parameter logistic model.

15. The method for creating a calibration curve according to claim 3, wherein the first approximate curve is created by regressing experiment data with a four-parameter logistic model.

16. The method for creating a calibration curve according to claim 4, wherein each of the first approximate curve and the second approximate curve is created by regressing experiment data with a four-parameter logistic model.

17. A fluorescence polarization immunoassay method in which an antibody, that has bindability to a target substance, and a fluorescently labeled substance, in which the target substance is labeled with a fluorescent dye are used to measure a concentration C_{Sβ} of the target substance in a sample to be measured β, the method comprising:
a step 4a of adding the antibody and the fluorescently labeled substance, in amounts equal to the added amounts in the step 1b, to the sample to be measured β, and measuring a degree of fluorescence polarization P_{Xβ} of the sample to be measured β; and
a step 4b, after the step 4a, of deriving, based on the degree of fluorescence polarization P_{Xβ}, the concentration C_{Sβ} of the target substance from the calibration curve created by the method according to claim 1.

18. A fluorescence polarization immunoassay device that uses an antibody, that has bindability to a target substance, and a fluorescently labeled substance, in which the target substance is labeled with a fluorescent dye to measure a concentration C_{Sβ} of the target substance in a sample to be measured β, the device comprising:
an emission optical system that emits linearly polarized excitation light on a sample;
a polarized light adjustment element that selectively allows, of fluorescence emitted from the sample, a linearly polarized component corresponding to a drive signal to pass through;
a receiver that detects fluorescence intensity that has passed through the polarized light adjustment element; and
a controller that outputs the drive signal to the polarized light adjustment element and measures, in accordance with the drive signal and based on the fluorescence intensity detected by the receiver, a degree of polarization of the sample,
wherein
the controller includes a storage medium that stores a program used in the fluorescence polarization immunoassay method according to claim 17, the program causing a computer to execute:
a step of deriving, based on the degree of fluorescence polarization P_{Xβ} of the sample to be measured β, the concentration C_{Sβ} of the target substance from the calibration curve.

19. A calibration curve creation kit comprising:
an antibody that has bindability to a target substance; and a fluorescently labeled substance in which the target substance is labeled with a fluorescent dye;
and further comprising:
at least one of a substance for removing the target substance from a solution containing the target substance, and a liquid not containing the target substance.
